(19) United States
(12) Patent Application Publication
(10) Pub. No.: US 2015/0328060 A1
(43) Pub. Date: Nov. 19, 2015

(54) ABSORBENT ARTICLE WITH INCREASED COMFORTABILITY

(71) Applicant: SCA HYGIENE PRODUCTS AB, Göteborg (SE)

(72) Inventors: Anna BAGGER-SJÖBÄCK, Göteborg (SE); Shabira ABBAS, Mölndal (SE); Angelica BURVALL, Bollebygd (SE); Lisa PALMQVIST, Mölndal (SE)

(73) Assignee: SCA HYGIENE PRODUCTS AB, Göteborg (SE)

(21) Appl. No.: 14/653,955

(22) PCT Filed: Dec. 20, 2012
(86) PCT No.: PCT/SE2012/051460

§ 371 Date: Jun. 19, 2015

(51) Int. Cl. A61F 13/535; A61F 13/475; A61F 13/537; A61F 13/47

(52) U.S. Cl. CPC A61F 13/535; A61F 13/4702; A61F 13/475; A61F 13/537; A61F 2013/15357

(57) ABSTRACT

An absorbent product with a plane extension a longitudinal direction, a transverse direction and a thickness direction is described. The product includes an absorption body, which includes, in its thickness direction, a liquid-receiving, open-cell foam layer and a liquid-absorbent fibrous layer. The liquid-receiving, open-cell foam layer has a total surface that covers the entire surface of the liquid-absorbent, fibrous layer in the plane extension; each of the longitudinally extending side edges extends at least along a part of its length outside of each of the longitudinally running side edges of the liquid-absorbent, fibrous layer; and has an air permeability that is greater than 200 m³/m²/min and an opacity greater than 35%. The liquid-absorbent, fibrous layer has a bending stiffness greater than 2.0 newtons measured according to the modified Circular Bend Procedure method.

### CROSS-REFERENCE TO PRIOR APPLICATION

This application is a §371 National Stage Application of PCT International Application No. PCT/SE2012/051460 filed Dec. 20, 2012, which is incorporated herein in its entirety.

### TECHNICAL AREA

The disclosure relates to an absorbent product such as a sanitary napkin, panty liner or an incontinence protector with a plane extension including a longitudinal direction, a transverse direction and a thickness direction, wherein the product includes an absorption body. The absorption body includes, in its thickness direction, a liquid-receiving, open-cell foam layer and a liquid-absorbent fibrous layer, wherein the liquid-receiving open-cell foam layer and the liquid-absorbent layer include two opposite, longitudinal side edges extending in the longitudinal direction and two opposite transverse edges extending in the transverse direction.

### BACKGROUND

For absorbent products such as sanitary napkins, panty liners and incontinence protectors that are provided to rest against the user's body when used, there are high requirements that they are discreet, soft and comfortable to wear and at the same time have a reliable security against leakage.

A common problem associated with absorbent products of this type is that the product deforms during use since the product is pressed together between the user's thighs. This can result in folds occurring in an uncontrolled manner in the product. The folds can result in channels that can form on the product surface and that the liquid runs out past the product's side edges and creates leakage. Furthermore, the compression of the product entails that the surface accessible for receiving liquid is reduced, whereby the risk that liquid ends up next to the product increases. The problem that undesired folds form commonly occurs, for example, when using airlaid, cellulose-based absorbent layers. In order to reduce the problem that the product deforms during use, in particular in the crotch part of the product, it proved to be advantageous to increase the stiffness of the product. Special materials with great stiffness, so-called stiffening elements, have been introduced into the product. Stiffening elements have the goal of retaining the shape of the product during use and of controlling the deformation in such a manner as to prevent the development of leakage. The stiffening elements can be substantially two-dimensional, three-dimensional or initially substantially two-dimensional but when the product is used they expand and form a three-dimensional structure. Documents that describe absorbent products comprising forming-and/or stiffening elements and able to retain their form under load are, for example, WO 98/22057, WO 98/22058, WO 98/22061 and WO 98/22062.

However, the skin in a user's crotch is relatively sensitive and a problem with using absorbent products with stiffening elements is that they can scrape the user's skin, for example, when the user moves. Furthermore, it is essential that the absorbent product is experienced to be reliable and that the user trusts that the product will function well as well as being soft and discreet to wear.

Furthermore, there is a risk that the user's skin becomes moist due to the fact that it becomes sealed around the user's genital area when using the absorbent product. It is a problem that the user experiences discomfort, and the risk of bacterial growth with accompanying skin and odour problems increases.

### SUMMARY

It is desired to create a product that is experienced as discreet, soft and comfortable to wear, while at the same time it exhibits satisfactory security against leakage has essentially been removed.

A first aspect includes a product comprising a liquid-receiving, open-cell foam layer having a total surface in the plane extension of the product that covers the entire surface of the liquid-absorbent, fibrous layer in the plane extension. The product further includes longitudinally extending side edges of the liquid-receiving, open-cell foam layer each extending at least along a part of its length outside of each of the longitudinally running side edges of the liquid-absorbent, fibrous layer. Also, the liquid-receiving, open-cell foam layer has an air permeability that is greater than 200 m³/m²/min and an opacity greater than 35%, and the liquid-absorbent, fibrous layer has a bending stiffness greater than 2.0 newtons according to the modified Circular Bend Procedure method. An advantage of the fact that the liquid-absorbent layer has a bending stiffness greater than 2.0 newtons is that this creates a relatively stiff and stable product, so that that the liquid-absorbent layer also functions as a stiffening element. The bending stiffness allows the product to not become so limp and that it cannot readily shrink together and create undesired folds in the crotch area. In order to simultaneously create a product that is soft, flexible and comfortable to wear, it proved beneficial that each longitudinal side edge of the liquid-receiving, open-cell foam layer extends at least along a part of its length outside of each longitudinally running side edge of the liquid-absorbent, fibrous layer, and that the liquid-receiving, open-cell foam layer has an air permeability that is greater than 200 m³/m²/min. The liquid-receiving foam layer is an open-cell, flexible and pliable structure and the foam's pliability and flexibility reduces the risk of scrapes on account of the stiff edge parts on the liquid-absorbent, fibrous layer. It turned out that liquid-receiving layers of airlaid, cellulose-based layers and liquid-receiving layers of non-woven material do not have the ability to reduce the negative effect of the stiff edges which a stiff cellulose-based absorption layer causes. Flexible foam materials can spring back, i.e., return to substantially their original shape after having been exposed to outer loading. Flexible foam materials also have a padding effect such that the foam material lines the stiff edges and creates a soft distancing element between the user's skin and the stiff edges of the liquid-absorbent layer.

Furthermore, the liquid-receiving foam layer is open-celled and in this manner retains a good liquid-receiving ability.

The liquid-receiving foam layer is an open-cell, continuous structure. Since the foam layer is a continuous structure, it exhibits good pliability and the ability to spring back following outer loading. On the contrary, fibre-and/or filament-based liquid-receiving layers include manifold discrete fibres and/or filaments that are often intermittently connected together to each other. However, the points where they are connected together do not create a continuous structure such as is created with a material formed by foam. Fibre- and/or filament-based layers therefore do not have as good an ability to spring back and resume their original form after an outer loading. Liquid-receiving open-cell and flexible foam layers have a good ability to resume their original form in all directions after an outer loading.

The pliability and the ability to spring back of the foam also brings it about that there is less risk for folds to be produced or that the layer shrinks in comparison to layers based on fibres and/or filaments.

The liquid-receiving, open-cell foam layer can include thermoplastic foam or hardenable foams (thermosetting foams). Examples of usable foams are polyolefin-based foam, polystyrene-based foam, PVC foam, polyvinyl alcohol foam, acrylate foam, for example, manufactured according to HIPE technology, polyurethane foam, epoxy foam, latex foam, urea-formaldehyde foam, melamine-formaldehyde foam, silicone foam, viscose foam, carboxymethyl cellulose (CMC) foam, starch form, chitosan foam, alginate foam, polyactide foam, polyglycolide foam and polycaprolactone foam.

The surface of the liquid-absorbent layer in the plane extension can have local differences in the bending stiffness. However, it is important that the bending stiffness over any area of the surface of the liquid-absorbent layer is greater than 2.0 newtons, i.e., the bending stiffness is greater than 2.0 newtons in accordance with the modified Circular Bend Procedure method in any area of the liquid-absorbent layer.

According to another embodiment, the surface of the liquid-absorbent layer in the plane extension has at least two different areas with a bending stiffness that is greater than 2.0 newtons according to the modified Circular Bend Procedure method. The surface, that has a bending stiffness that is greater than 2.0 newtons according to the modified Circular Bend Procedure method, should be able to be measured on two different areas on the surface of the liquid-absorbent layer, wherein two areas can be located adjacent to one another or be placed separated from one another.

According to another embodiment, the liquid-absorbent layer has a bending stiffness that is greater than 2.0 newtons according to the modified Circular Bend Procedure method over its entire surface.

It has proved to be an advantage, especially for sanitary napkins that are to absorb menstrual liquid, which has a strong colour, that the absorbed liquid that is absorbed into the product is not very visible. Due to the fact that the liquid-receiving, open-cell foam layer has an opacity that is greater than 35%, it turned out that the user experiences the product as more pleasing than a product that has liquid-receiving layers that are not as opaque, for example, fibre layers of airy, non-woven material. It is also important that the user finds, before using the product, that it is a reliable and aesthetically pleasing product. Due to the fact that the liquid-receiving layer has an opacity that is greater than 35%, the difference in size of the layers in the absorption body is not seen as distinctly, which gives a more reliable impression of the product even before use.

According to another embodiment, the liquid-receiving, open-cell foam layer has an opacity greater than 50%.

According to an embodiment, each longitudinal side edge of the liquid-receiving foam layer extends at least 5.0 millimetres outside of each longitudinally running side edge of the liquid-absorbent layer. The width of the surface of the liquid-receiving foam material in the longitudinal direction of the product extending outside of the surface of the underlying liquid-absorbent layer should be adapted in such a manner that a good flexibility is obtained. In certain embodiments, the width on each longitudinally running side edge of the liquid-receiving foam layer extending outside of the underlying absorbent layer is between 5-15 mm. As was previously described, the flexible foam material has a padding effect such that the foam material lines the stiff edges. In order to obtain a good padding effect it turned out, at least in some instances, that this distance, 5-15 mm, is optimal. As was also described previously, the ability of the foam material to spring back, i.e., to return to substantially its original form after having been exposed to outer loading is also an important property. The foam material has the ability to regain its original form in all directions, not only in the direction of the thickness of the material.

According to an embodiment, each longitudinally running side edge of the open-cell, liquid-receiving foam layer extends outside of each longitudinally running side edge of the liquid-absorbent, fibrous layer along its entire length. An advantage of such an embodiment is that such a product is experienced as softer, airier and also as more aesthetically pleasing by some users.

According to an embodiment, the total surface of the open-cell, liquid-receiving layer in its plane extension is at least 1.7 times as great as the total surface of the liquid-absorbent, fibrous layer in the plane extension of the product. At least in this embodiment, it turned out that the total surface of the liquid-receiving layer in its plane extension should be at least 1.7 times as great as the surface of the liquid-absorbent layer in order to obtain a product with optimal airiness. For some products, for example for a panty liner, that does not need to be able to absorb a great volume of menstrual liquid, the total surface of the liquid-absorbent, fibrous layer does not need to be equally great, which brings it about that for such products it can be advantageous if the total surface of the liquid-receiving foam layer in its plane extension is at least 2.0 times as great as the total surface of the liquid-absorbent, fibrous layer in the plane extension of the product. By virtue of the fact that the open-cell, liquid-receiving foam layer has an opacity that is greater than 35%, the difference in size on the layers is not seen as clearly, which makes the product more aesthetically pleasing and gives the product a more reliable impression. Therefore, a high opacity of the liquid-receiving layer is especially advantageous for products with a large difference of the surface between the liquid-receiving layer and the liquid-absorbent layer.

A small surface of the liquid-absorbent layer can be desirable for certain products, in part for making them thin and discreet and in part for reducing the material cost. Furthermore, an advantage with a large difference on the surface between the liquid-receiving layer and the liquid-absorbent layer is that when the liquid-absorbent layer also functions as a shaping element that follows the body when it moves, a certain difference of the layers can be required for obtaining the desired shaping.

According to another embodiment, the open-cell liquid-receiving foam layer has an air permeability that is at least 300 m³/m²/min. Such an air-permeable, open-cell liquid-receiving foam layer reduces the risk that the product is experienced as tight and uncomfortable.

According to another embodiment, the liquid-absorbent fibrous layer has a bending stiffness that is greater than 3.0 newtons according to the modified Circular Bend Procedure method. The liquid-absorbent layer falls within this embodiment since the bending stiffness is greater than 3.0 newtons according to the modified Circular Bend Procedure method in any area of the liquid-absorbent fibrous layer, i.e. in at least one area of the liquid-absorbent fibrous layer.

According to yet another embodiment, the liquid-absorbent fibrous layer has a bending stiffness that is greater than 4.0 newtons according to the modified Circular Bend Procedure method. The liquid-absorbent layer falls within this embodiment since the bending stiffness is greater than 4.0 newtons according to the modified Circular Bend Procedure method in any area of the liquid-absorbent fibrous layer, i.e. in at least one area of the liquid-absorbent fibrous layer.

According to another embodiment, the open-cell liquid-receiving foam layer has a bending stiffness that is less than 0.3 newtons according to the modified Circular Bend Procedure method. The liquid-receiving open-cell foam layer falls within this embodiment since the bending stiffness is less than 0.3 newtons according to the modified Circular Bend Procedure method in any area of the liquid-receiving open-cell foam layer, i.e. in at least one area of the liquid-receiving open-cell foam layer.

According to another embodiment, the open-cell liquid-receiving foam layer has a bending stiffness that is less than 0.25 newtons according to the modified Circular Bend Procedure method. The liquid-receiving layer falls within this embodiment since the bending stiffness is less than 0.25 newtons according to the modified Circular Bend Procedure method in any area of the liquid-receiving layer, i.e. in at least one area of the liquid-receiving layer.

According to an embodiment, the absorbent product comprises a liquid-permeable surface material and a backsheet that is liquid-tight, wherein the absorption body is arranged between the liquid-permeable surface material and the liquid-tight backsheet, and that the liquid-receiving foam layer is placed against the liquid-permeable surface material and the liquid-absorbent, fibrous layer is placed against the liquid-tight backsheet.

However, it is also possible that the absorbent product does not have a separate liquid-permeable surface material. In such an embodiment, the liquid-receiving layer is placed closest to the user when the product is being used, and thus the surface closest to the user consists of a flexible foam. An advantage of such an embodiment, is that the surface closest to the user is soft, smooth and pliable and that the number of layers used in the product is less, which simplifies the manufacturing process and possibly also reduces the material cost.

The liquid-absorbent, fibrous layer can extend in the longitudinal direction of the product over the crotch part and at least in part over the front part. In the transition between the crotch part and the front part, the liquid-absorbent, fibrous layer can have a width (M) which is less than 40 mm. Furthermore, the side edges of the liquid-absorbent, fibrous layer diverge in the front part of the product in the direction from the crotch part to at least a little over the front part. The width (M) can be between 25-40 mm, or between 30-35 mm. The narrowest point between the user's legs is where the muscle group passes that has its origin on the inside of the bottom of the pelvis and its attachment along the thigh. This muscle group consists of the muscles adductor brevis, adductor longus and adductor magnus. Measurements show that the distance in the user's crotch area between this adductor muscle group on the left and right side is surprisingly similar for all people and is around 30 mm. Since the liquid-absorbent layer of the product is constructed in a relatively stiff fibrous material, the width of the crotch part on the liquid-absorbent layer, at least in the area that is to pass between the muscle groups of each leg, should not exceed 40 mm, therefore. A width (M) that is less than 40 mm also reduces the risk of undesirable fold formation in the liquid-absorbent, fibrous layer.

By designing the product with a sharply tapering part from the widest extension of the front part in the transverse direction of the product to the transition M between the front part and the crotch part, more transverse edges are obtained, see FIG. 4, which cling to the user's legs during use and prevent the product from gliding backwards. To obtain a good clinging effect, it proved suitable that the front part tapers from its full width to a width corresponding to the smallest available width in the user's crotch area in 20 mm of the product's length.

The absorbent product can be a sanitary napkin, a panty liner or an incontinence protector.

The product can be provided with a fastening element for fastening the product in a pair of pants. Suitable fastening elements are adhesive, for example, Velcro fastening surfaces, friction coatings or the like. The fastening element can also comprise side tabs or wings. Such side tabs or wings are folded around the crotch of the pants and attached to the pants or to one another.

### BRIEF DESCRIPTION OF THE FIGURES

Embodiments of the invention will be described in detail in the following with reference made to the exemplary embodiments shown in the attached drawings. In the figures:

FIG. 1 shows a sanitary pad viewed from above;

FIG. 2 shows a section along line II-II through the sanitary napkin in FIG. 1.

FIG. 3 shows a section along line II-II through an alternative embodiment of the sanitary napkin in FIG. 1.

FIG. 4 shows another alternative embodiment of the sanitary napkin in FIG. 1 viewed from above.

### DESCRIPTION OF PARTICULAR EMBODIMENTS

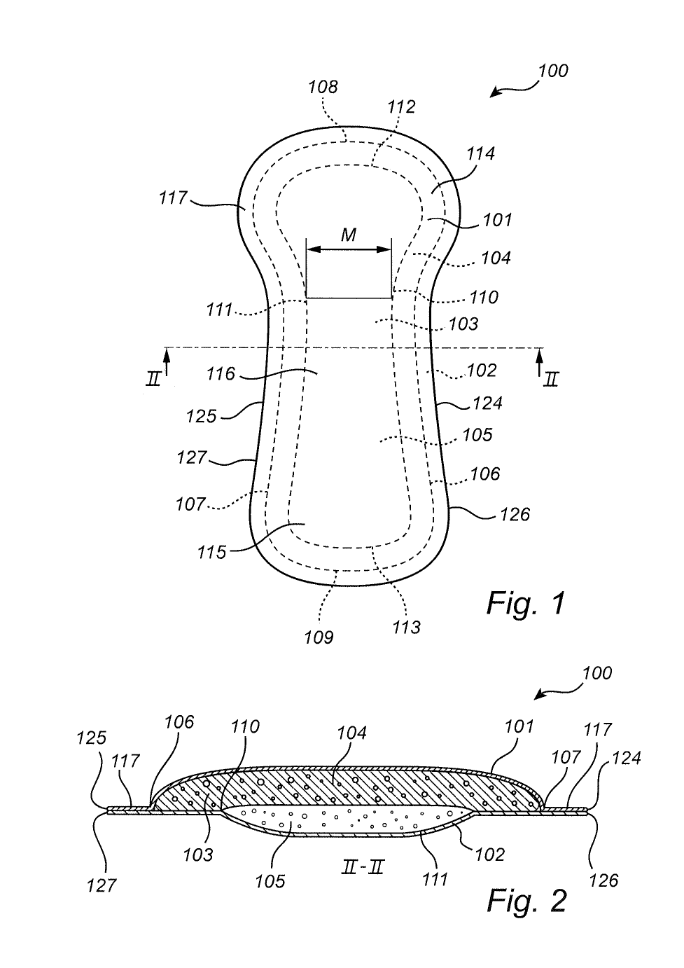

The sanitary napkin 100 shown in the FIGS. 1 and 2 is elongated with a longitudinal direction and a transverse direction. The sanitary napkin 100 includes a liquid-permeable surface material 101 arranged on the top side of the sanitary napkin, i.e., the side of the sanitary napkin intended to be turned toward a user during use, a liquid-tight backsheet 102, and an absorption body 103 arranged between the surface material 101 and the backsheet 102. The absorption body 103 includes a liquid-receiving, open-cell foam layer 104 placed against the liquid-permeable surface material 101 and a liquid-absorbent fibrous layer 105 placed against the liquid-tight backsheet 102, whereby the liquid-receiving, open-cell foam layer 104 includes two opposing, longitudinally running side edges 106, 107 extending in the longitudinal direction and two opposing transverse edges 108, 109 extending in the transverse direction, and the liquid-absorbent, fibrous layer 105 includes two opposing longitudinally running side edges 110, 111 extending in the longitudinal direction, and two opposing transverse edges 112, 113 extending in the transverse direction. The liquid-receiving, open-cell foam layer 104 has a total surface in the plane extension of the product that covers the entire surface of the liquid-absorbent, fibrous layer 105 in its plane extension, and each longitudinally running side edge 106, 107 of the liquid-receiving foam layer 104 extends at least along a part of its length outside of each longitudinally running side edge 110, 111 of the liquid-absorbent, fibrous layer. Furthermore, the liquid-receiving open-cell foam layer 104 has a permeability to air that is greater than 200 m³/m²/min and an opacity that is greater than 35%. The liquid-absorbent, fibrous layer 105 has a bending stiffness that is greater than 3.0 newtons.

The surface material 101 and the backsheet 102 have substantially the same plane form as the absorption body 103 but have a somewhat greater extension in the plane, as a result of which they form a projecting edge 117 around the entire periphery of the absorption body 103. Cover layers 102, 103 are mutually connected inside the projecting edge 117, for example, by gluing, sewing or welding with heat or ultrasound.

The sanitary napkin in FIG. 1 is formed with a front part 114, a back part 115 and an intermediate crotch part 116. The front part 114 has a rounded form and is wider than the crotch part 116. It turned out that all users have a critical area between the groins where the distance between the muscles running down on the inside of the thighs is approximately 30-35 mm. The width of the sanitary pad in the crotch area is limited in front by this distance between the muscle sinews right in front of the users' groins. Thus, in the transition between the crotch part 116 and the front part 114 the liquid-absorbent layer 105 has a distance M that does not exceed 40 mm and, in particular embodiments, is 30-35 mm. For the sanitary napkin 100, this is the width of the liquid-absorbent layer 105 in the transition between the crotch part 116 and the front part 114 that should not be too wide. The reason it is the width of the liquid-absorbent layer 105 in the crotch part 116 of the product which is most important is that it is this layer that contributes to the product's stiffness. In the direction back from this transition with width M to the end of the crotch part the width of the liquid-absorbent, fibrous layer 105, which also functions as a stiffening element, can continuously increase to a magnitude of size 1.5 times the width M.

A relatively narrow distance M, at least on the liquid-absorbent layer 105, is also an advantage from the viewpoint of fastening, since a sanitary pad where the difference in width between the front part 114 and the width of the narrowest part on the crotch part 116 is large, yields a good fastening effect against the user's legs and prevents the sanitary pad from gliding backwards during use.

The liquid-permeable surface material 101 suitably includes a conventional liquid-permeable material. Examples of suitable materials are perforated plastic films, non-woven materials, plastic nets or the like.

The liquid-tight backsheet 102 is a conventional type and can include any liquid-tight material suitable for the purpose. Examples of such materials are various types of thin plastic films or non-woven materials treated to resist the penetration of liquid, for example, by being coated with plastic, wax or the like. Even other treatments such as heat calendering for melting a material that was permeable in the beginning to a mainly liquid-tight layer can be used. Furthermore, the liquid-tight backsheet 103 can include a liquid-tight surface on absorption body 103. In order to produce an airy product, it is common to use liquid-tight backsheet material that is breathable, i.e., has a good air permeability. According to embodiments, the liquid-tight backsheet material includes a breathable material. Examples of breathable materials are perforated films, microporous films, macroporous films, nanoporous films, monolithic films, fibrous non-woven ones and their laminates.

An example of a liquid-absorbent, fibrous layer 105 with a high absorption capacity and a good capacity for transporting liquid is the fibrous material described in WO 94/10953 and WO 94/10956. These materials are present in the form of dry-formed fibre layers with a high density and stiffness and are used directly in an absorbent product without being defibrated at first. The stiffening and absorbent element can also be made from a laminate of several non-woven layers or tissue layers that are mutually fixed for increased stiffness and that have highly absorbent particles between individual layers. The fixing of the individual layers to each other can take place with binding agents such as adhesive or melted fibres. The highly absorbent particles can also contribute to the bonding. The stiffness is controlled by selecting the number of layers and the amount of binding agent used and by the selection of highly absorbent material and how its adhesive capacity is utilized.

Another example of material in the liquid-absorbent layer is one or more layers of airlaid cellulose layers. This can be prefabricated material which is supplied in roll form for being then cut out in a suitable size, or alternatively the liquid-absorbent layer can be mat-formed and formed in-line during the manufacture itself of the absorbent product.

For example, to obtain compression lines/compression zones, there are areas that are compressed at a higher bending stiffness than the surrounding areas. Such a further compression can take place in combination with the compression of the liquid-absorbent layer. Alternatively, a pattern compression can take place in a separate step after the flat compression. The compression of the liquid-absorbent, fibrous layer can be made in several ways. An example of a common process is one that is called “high-density compression” (HDC), which is described in detail in EP-B-1427658. The compression can be made in two to three steps that include a preliminary compression and thereafter a compression in one or two steps. It is also possible to perform the compression in one step.

The surface of the liquid-absorbent layer in plane extension can exhibit local differences in bending stiffness. However, in certain embodiments, the bending stiffness over one area on the surface of the liquid-absorbent layer is greater than 2.0 newtons, i.e., the liquid-absorbent layer has a bending stiffness greater than 2.0 newtons according to the modified Circular Bend Procedure method in any area of the liquid-absorbent layer.

According to another embodiment, the surface of the liquid-absorbent layer in its plane extension includes at least two different areas with a bending stiffness that is greater than 2.0 newtons according to the modified bending stiffness method. It includes the fact that the surface that has a bending stiffness greater than 2.0 newtons according to the modified Circular Bend Procedure method should be able to be measured on two different areas on the surface of the liquid-absorbent layer, which two areas can be adjacent to one another or can be placed at a distance from one another.

The liquid-absorbent layer can have a bending stiffness greater than 2.0 newtons according to the modified Circular Bend Procedure method over the entire liquid-absorbent layer. According to this embodiment, no area on the liquid-absorbent layer may have a bending stiffness that is 2.0 newtons or lower when measured according to the modified Circular Bend Procedure method.

A suitable liquid-receiving, open-cell foam layer 104 is polyurethane foam. Polyurethane is a two-component product consisting of polyol and isocyanate that are mixed to polyurethane foam. Polyurethane has either open or closed cells. For being used as a liquid-receiving foam layer in an absorbent product, polyurethane foam is used with open cells. Polyurethane foam can have different stiffness and for our purpose flexible foams are used. The liquid-receiving foam is flexible with a low bending stiffness and springs back well, i.e., after being loaded the foam returns to substantially its original form.

The liquid-receiving, open-cell foam layer can be made of thermoplastic foam or thermosetting foams. Examples of usable foams are polyolefin-based foam, polystyrene-based foam, PVC foam, polyvinyl alcohol foam, acrylate foam, for example, manufactured according to HIPE technology, polyurethane foam, epoxy foam, latex foam, urea-formaldehyde foam, melamine-formaldehyde foam, silicone foam, viscose foam, carboxymethyl cellulose (CMC) foam, starch foam, chitosan foam, alginate foam, polyactide foam, polyglycolide foam and polycaprolactone foam.

A fastening element in the form of a rectangular area extending in the longitudinal direction and made of self-adhesive glue is arranged on the outside of the liquid-tight backsheet layer. When the sanitary napkin 100 is being used, it is placed inside the user's underpants and is fastened in the underpants with the aid of the fastening element. Before being used, the fastening element is protected in a conventional manner, for example, by being covered by a protective layer of paper or plastic treated with silicone, or is embossed so that it can be readily separated from the glue when the sanitary napkin is to be used. The glue can obviously be alternatively arranged in any pattern suitable for the purpose such as a plurality of longitudinally running strands, an entire covering, arranged in areas only at the front part and/or the back part or the like. Furthermore, other types of fastening elements can be used such as friction covering, press studs, clamps, fastening flaps or the like.

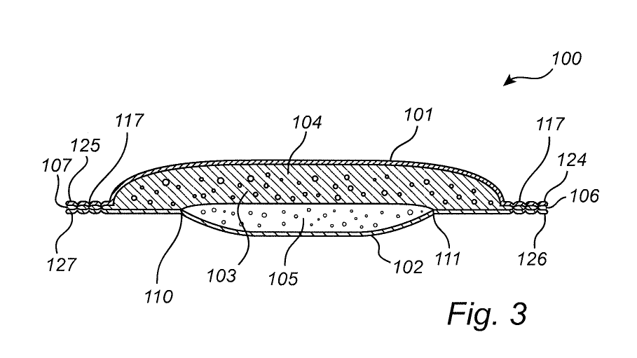

FIG. 3 shows a transverse section of an alternative embodiment of the sanitary napkin 100. The liquid-receiving foam layer 104 has a total surface in the plane extension of the product that is substantially the same as the surface of the liquid-permeable surface material 101 and substantially the same as the surface of the liquid-tight backsheet material 102. The liquid-permeable surface material 101 includes two opposing, longitudinally running side edges 124, 125 extending in the longitudinal direction and the liquid-tight backsheet material 102 includes two opposing longitudinally running side edges 126, 127 extending in the longitudinal direction. Thus, the two opposing longitudinal side edges 106, 107 of the open-cell, liquid-receiving foam layer 104, which side edges extend in the longitudinal direction, coincide substantially with the longitudinally running side edges 124, 125 of the liquid-permeable surface material and the longitudinally running side edges 126, 127 of the liquid-tight backsheet material. Thus, the surface material 101, backsheet material 102 and the liquid-receiving foam layer 104 include a projecting edge 117 around the entire periphery of the liquid-absorbent, fibrous layer 105. Cover layers 102, 103 and the liquid-receiving foam layer 104 are mutually connected inside the projecting edge 117, for example, by gluing, sewing or welding with heat or ultrasound. Therefore, the surface of the liquid-receiving foam layer 104 extends in its plane extension outside of each longitudinally running side edge 110, 111 of the liquid-absorbent, fibrous layer 105 and of each transversely running side edge 112, 113 of the liquid-absorbent, fibrous layer 105.

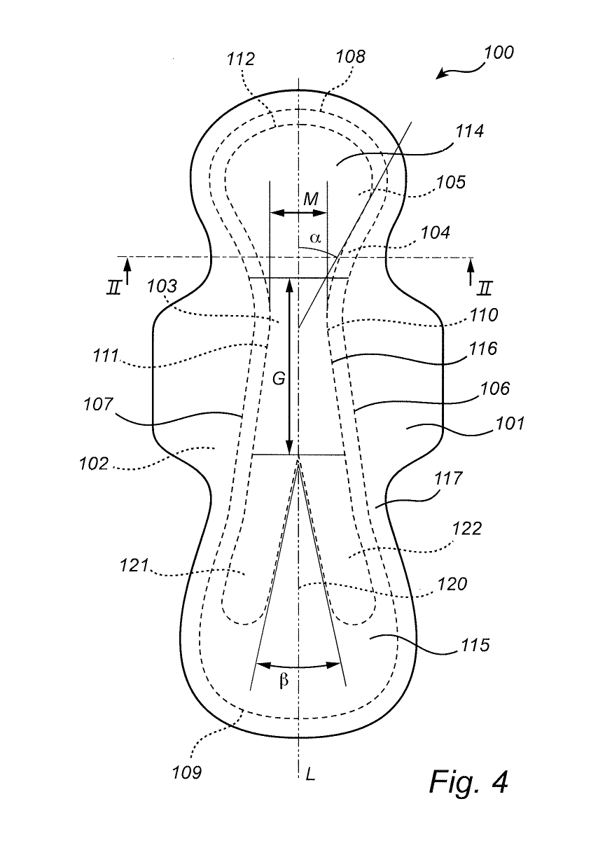

Furthermore, FIG. 4 shows an alternative embodiment of the sanitary napkin 100. FIG. 4 shows the sanitary napkin from the top. The liquid-absorbent, fibrous layer 105 includes a shape like a keyhole. The liquid-receiving foam layer 104 also has a shape like a keyhole but with a greater extension in the longitudinal direction as well as in the transverse direction than the liquid-absorbent, fibrous layer 105. The liquid-absorbent, fibrous layer 105 is also intended to function as a stiffening element and is designed to reduce the risk that the sanitary napkin deforms in an uncontrolled manner. The liquid-absorbent, fibrous layer 105 has a size, shape and stiffness that bring it about that the product retains during the entire time of use a predetermined shape and furthermore is held fast at the intended location on the user.

The liquid-absorbent layer has a width M in the transition between the crotch part 116 and the front part 114 which width is less than 40 mm, or 30-35 mm. Both side edges of the front part 114 diverge in a forward direction in the product from this transition M. In this manner, it is prevented that the product is shifted backwards between the user's legs. In FIG. 4, there is an angle between a line in the longitudinal direction of the product and each of the side edges of the liquid-absorbent, fibrous layer in the front part designated with α. In the case of a large angle α, for example, in the vicinity of 90°, the edges of the front part can scrape against the users groins and legs, thus creating discomfort for the user. The smaller the angle α is, the greater the risk that the product slides backwards in between the user's legs. At an angle below 30° this risk is unacceptably great. An angle between 35-45° gives the best balance between a firm hold and comfort.

The sanitary napkin 100 in FIG. 4 is formed with a crotch length adapted to the user's anatomy. In sanitary napkin described herein, the fact was utilized that most women have a crotch length with a size of 80-100 mm. Therefore, the liquid-absorbent, fibrous layer 105 was created with a corresponding crotch length G with the size of 70-120 mm. Along the crotch, where the user's body shape is substantially plane, the sanitary napkin is shaped so as to be relatively stiff laterally, i.e., it is sufficiently stiff so as to not be deformed laterally and form folds. Since it is important that the liquid-absorbent, fibrous layer has a good absorption capacity, it is important to be able to utilize accessible space between the user's legs in the crotch. The width of the sanitary pad in the crotch area is limited in front by this distance between these muscle sinews right in front of the user's groins. In the direction backwards from these transition areas to the end of the crotch part the width of the liquid-absorbent, fibrous layer 105, that also functions as a stiffening element, can continuously increase to the size of 1.5 times the width M without the risk that the liquid-absorbent layer 105 scrapes the user in the crotch.

The liquid-absorbent layer 105 extends slightly over the product's back part 115. The liquid-absorbent layer 105 has a recess 120 in the back part 115 and extending from its end edge and in the direction toward the crotch part 116 by means of which the product can fold along a longitudinally running line in the recess and by means of which the parts, the legs 121 and 122, that are located on both sides of the recess 120, become more flexible than the wider crotch part 116. This recess 120 is for obtaining a good adaptation to and pliability with the body. The fold which develops in the recess when the product is being used can pass into the user's posterior notch, thus offering a very good protection against leakage via the posterior notch, which type of leakage occurs when using sanitary pads when the user is lying on her back. Furthermore, the recess 120 makes it possible that these legs 121, 122 can be mutually shifted vertically during various body movements, for example, when the user is walking.

In the exemplary embodiment shown in FIG. 4, the recess 120 is wedge-shaped and symmetrically located relative to the longitudinally running line of symmetry L of the product and forms an angle β with a magnitude of 20°. This angle can vary within broad limits but is of course dependent on the shape of the back part 115.

Embodiments of the invention have been described above in connection with a sanitary napkin. It is possible, however, to utilise the invention also for panty liners and incontinence protectors.

### EXAMPLES

The following materials were tested:

1. Polyurethane foam, FXI Foamex Innovations Inc., product code CAZ80A

2. Polyurethane foam, Foamex Innovations Inc., product code C80H2A

3. Polyurethane foam, FoamPartner, Reisgies Schaumstoffe GmbH, Regilen 30WF, item number 190321

4. Polyurethane foam, Caligen Foam Ltd., product code E 50

5. Melamine-based foam, BASF Plastics, Basotect W., product code E 2419 10

6. Polyurethane foam, Woodbridge Foam, product code SM25WH

7. Carded through, air-bound, non-woven, Fiberweb Tenotex, product code Airten 1250W6

8. Cellulose-based, multi-bound airlaid, Glatfelter Falkenhagen GmbH, product code MH080.137

9. Cellulose-based airlaid without SAP, “HDC” manufactured according to EP 1427658B1

10. Cellulose-based airlaid with 25 weight percent SAP, “HDC” manufactured according to EP 1427658B1

11. Cellulose-based, multi-bound airlaid with 32 weight percent SAP, Glatfelter Falkenhagen GmbH, product code VF250.103

12. Cellulose-based airlaid without SAP, manufactured according to WO 94/10953 with embossing pattern number 2072

13. Cellulose-based airlaid with 10 weight percent SAP, manufactured according to WO 94/10953 with embossing pattern “Wave”.

Explanations:

SAP=Super-absorbent particles manufactured from cross-bound and partially neutralized acrylic acid

Multi-bound airlaid consists primarily of cellulose fibres that are bound with tex bi-component molten fibres and binding agent of ethylene vinylacetate copolymer “HDC”=High Density Compression is in the example a highly compressed, defibrated and mat-formed chemical sulphate mass with or without SAP

The materials 1-8 in examples 12 and 13 are based on fling-dried CTMP mass with or without SAP

Materials 1-8 relate to liquid-receiving layers and materials 9-13 relate to liquid-absorbent layers. In the liquid-receiving layers 1-8 materials 1-6 relate to liquid-receiving, open-cell foam layers and materials 7 and 8 relate to liquid-receiving, fibrous layers. In the liquid-absorbent layers 9-13 all relate to liquid-absorbent, fibrous layers.

### Density

The measurements were carried out in accordance with the EDANA method WSP 130.1.

In order to measure the average density of a material layer in an absorbent product it is essential that the various material layers included in the absorbent product are separated with caution. The material specimen to be measured is clipped out of the material layer. A loading pressure of 0.5 kPa is applied to the material specimen by a foot with an area of 45×45 mm and that is less than the area of the material specimen. Then the thickness of the material specimen is measured. The basis rate of the material sample, i.e., gram/cm², is weighed and calculated forward. Then, the density of the material specimen is calculated by dividing the base rate by the thickness.

Result:

| Material | Thickness | Density (kg/m³) |
|---|---|---|
| 1 | 2.0 | 31 |
| 2 | 2.0 | 29 |
| 3 | 2.0 | 29 |
| 4 | 2.8 | 31 |
| 5 | 1.7 | 10 |
| 6 | 2.1 | 23 |
| 7 | 1.6 | 30 |
| 8 | 1.3 | 65 |
| 9 | 2.5 | 205 |
| 10 | 1.6 | 300 |
| 11 | 1.4 | 216 |
| 12 | 1.0 | 350 |
| 13 | 0.8 | 330 |

Air permeability

The measurements were carried out according to the EDANA method WSP 70.1. The apparatus used was the Textest FX 3300 from Testex Instruments. The apparatus measures what air permeability a material has in a defined pressure drop. We measured at a pressure drop of 200 Pa. The apparatus includes a pump that sucks air through a hole and a measuring arm. The size of the hole is 20 cm². The air flow through the material is measured.

Result:

| Material | Air permeability |
|---|---|
| 1 | 34 |
| 2 | 164 |
| 3 | 222 |
| 4 | 462 |
| 5 | 333 |
| 6 | 438 |
| 8 | 117 |

The value relates to the mean value from 10 measurements. The instrument's function was checked prior to the measurements against a standardised rubber plate that has a tightness of 104 l/m²/sec at 200 Pa. The standardised rubber plate is available as an accessory from the instrument manufacturer.

### Stiffness

Method Description for Modified Circular Bend Procedure:

The method for the modified Circular Bend Procedure was carried out according to a modified version of ASTM D 4032-82 (Circular Bend Procedure). The method for the modified Circular Bend Procedure is described in detail in EP 336 578. The measuring was carried out in the same manner as described in EP 336 578.

The apparatus that was used is the Instron Model No 5965. Instron Model No 5965 is manufactured by Instron Engineering Corporation. The bending resistance in the material sample was measured by measuring the maximum bending stiffness. According to the method, the maximum bending stiffness is constituted by a simultaneous deformation in several directions of a material sample, wherein one of the surfaces of the material sample becomes concave and the other surface of the material sample becomes convex. The method for the modified Circular Bend Procedure supplies a force value that concerns the bending resistance, i.e. simultaneous medium stiffness in all directions. The equipment used for the method of the modified Circular Bend Procedure is a modified Circular Bend Stiffness Tester that has the following parts:

A smooth-polished steel plate with measurements 102.0×102.0×6.35 mm that has a circular opening with a diameter of 18.75 mm. The nozzle has a beveled edge that is 45 degrees to a depth of 4.75 mm. A pressure rod with a total length of 72.2 mm, a diameter of 6.25 mm and a spherical end with a radius of 2.97 mm was used. A sharp needle tip projects 0.88 mm out from the spherical end. The pressure rod was mounted concentrically and has the same magnitude of play in all directions. The end of the pressure rod is placed well over the plate with the circular opening. From this position the downwardly directed strike of the spherical tip is so long that it precisely extends to the bottom of the nozzle of the plate. However, an indication of the distance of the needle tip (0.88 mm) is not included. A tester of draught and compression with a loading cell adapted for Instron Model No. 5965 was used.

Production of Test Materials and Calculation of the Average Value:

The liquid-receiving-respectively the liquid-absorbent material layer from five absorbent products was measured and then the average value was calculated.

In those cases where the liquid-absorbent layer includes areas with different bending stiffness, for example, an area that is more compressed than an adjacent area, the liquid-absorbent layer falls within the scope of protection since any area has a bending stiffness that is greater than 2.0 newtons.

In those cases where the liquid-receiving foam layer includes areas with different bending stiffness, for example, an area that is more compressed than an adjacent area, the liquid-receiving foam layer falls in a corresponding manner within the scope of protection since any area has a bending stiffness that is lower than that which is indicated in the claim.

When measuring material layers in an absorbent product, it is essential that the liquid-receiving foam layer is separated from the liquid-absorbent layer with great care so that the material layers do not break into pieces during the separating.

Implementation:

The implementation was made exactly in accordance with the description in EP 0 336 578 Al. The material specimens are stamped out or cut out and have an area of 37.5×37.5 mm. The material specimens were conditioned for two hours at a temperature of 21±1° C. and a relative air humidity of 50±2%. The pressure rod should be moved downward at a rate of 50.0 cm/min.

The material specimen is then placed centred over the opening of the steel plate. The surface of the material specimen that is turned in the product towards the liquid-permeable surface material was turned in the taking of the specimen towards the pressure rod and the surface of the material sample that was turned in the product towards the liquid-impervious backside material was turned in the taking of the specimen towards the steel plate. The pressure plate was then put in motion and the maximum force was measured. The value of the maximum force was rounded off to the nearest gram.

Result:

| Material | Bending stiffness (newtons) |
|---|---|
| 1 | 0.29 |
| 2 | 0.16 |
| 3 | 0.20 |
| 4 | 0.10 |
| 5 | 0.27 |
| 6 | 0.18 |
| 7 | 0.07 |
| 8 | 0.59 |
| 9 | 3.43 |
| 10 | 2.28 |
| 11 | 3.40 |
| 12 | 6.55 |
| 13 | 2.94 |

Materials 1-8 are liquid-receiving layers. The result shows that layers 1-6 that are open-cell foam materials and also material 7, that is a carded through air-bound nonwoven material, have a bending stiffness that is lower than 0.30 newtons. In contrast thereto, material 8, which is a cellulose-based, multi-bound airlaid, exhibits a bending stiffness that is greater than 0.30 newtons.

Materials 9-13 are liquid-absorbent layers that exhibit a bending stiffness greater than 2.0 newtons.

The measurements were carried out in accordance with EDANA's method WSP 60.1.

Opacity is a concept and a magnitude in optics that is used to indicate transparency, that is, translucency. Opacity measures the degree of impenetrability for radiation through a material or transmission medium. Material that entirely lacks the ability to let light through is called opaque. The opacity was measured with a spectrophotometer with measurement in the YXY colour system (CIE 1931). The spectrophotometer used was of the brand Minolta Chroma Meter CR 300 (the y value was used) (CIE Illuminant D65). Calibration plate No. 16133079 was used as white standard background with a reflectance of 0.89. Black velvet was used as black standard background with a reflectance of 0.005.

The implementation was made in accordance with EDANA's method WSP 60.1.

The opacity (contrast ratio C_{0.}89) is calculated as follows:

Contrast ratio C_{0.}89=R_{b}/R_{w}×100

R_{b}=brightness, a specimen piece against black standard background

R_{w}=brightness, a specimen piece against white standard background

The results are indicated with accuracy in whole numbers.

Result:

| Material | Opacity (contrast ratio C_{0.}89, %) |
|---|---|
| 1 | 57 |
| 2 | 37 |
| 3 | 39 |
| 5 | 48 |
| 6 | 37 |
| 7 | 67 |
| 8 | 31 |

The result shows that the foam materials, i.e., materials 1, 2, 3, 5 and 6 have an opacity above 35%. Material 7, which is a cellulose-based material, also has an opacity that is over 35%. In contrast thereto, material 8, that is a carded through, air-bound non-woven material has a lower opacity, 31%.

Foam materials 1, 2, 3, 5 and 6, as well as fibres/filaments in materials 7 and 8 are substantially white. However, it is also possible to have the materials in the layers coloured in other colours.

1. An absorbent product with a plane extension having a longitudinal direction, a transverse direction and a thickness direction, the product comprises an absorption body, which absorption body comprises, in its thickness direction, a liquid-receiving, open-cell foam layer and a liquid-absorbent fibrous layer,
wherein the liquid-receiving open-cell foam layer and the liquid-absorbent fibrous layer comprise two opposite, longitudinally running side edges extending in the longitudinal direction and two opposite transverse edges extending in the transverse direction,
wherein the liquid-receiving, open-cell foam layer has a total surface in the plane extension of the product that covers the entire surface of the liquid-absorbent, fibrous layer in the plane extension,
wherein each of the longitudinally extending side edges of the liquid-receiving, open-cell foam layer extends at least along a part of its length outside of each of the longitudinally running side edges of the liquid-absorbent, fibrous layer,
wherein the liquid-receiving, open-cell foam layer has an air permeability that is greater than 200 m³/m²/min and an opacity greater than 35%, and
wherein the liquid-absorbent, fibrous layer has a bending stiffness greater than 2.0 newtons measured according to the modified Circular Bend Procedure method.

2. The absorbent product according to claim 1, wherein each longitudinally running side edge of the liquid-receiving, open-cell foam layer extends at least 5.0 millimetres outside of each longitudinally running side edge of the liquid-absorbent, fibrous layer.

3. The absorbent product according to claim 1, wherein each longitudinally running side edge of the liquid-receiving, open-cell foam layer extends outside of each longitudinally running side edge of the liquid-absorbent, fibrous layer along its entire length.

4. The absorbent product according to claim 1, wherein the total surface of the liquid-receiving, open-cell foam layer in its plane extension is at least 1.7 times as great as the total surface of the liquid-absorbent, fibrous layer in the plane extension of the product.

5. The absorbent product according to claim 4, wherein the total surface of the liquid-receiving, open-cell foam layer in its plane extension is at least 2.0 times as great as the total surface of the liquid-absorbent, fibrous layer in the plane extension of the product.

6. The absorbent product according to claim 1, wherein the liquid-receiving, open-cell foam layer has an air permeability that is at least 300 m³/m²/min.

7. The absorbent product according to claim 1, wherein the liquid-absorbent, fibrous layer has a bending stiffness that is greater than 3.0 newtons.

8. The absorbent product according to claim 1, wherein the liquid-receiving, open-cell foam layer has a bending stiffness that is less than 0.30 newtons.

9. The absorbent product according to claim 1, wherein the liquid-receiving, open-cell foam layer (105) has an opacity greater than 50%.

10. The absorbent product according to claim 1, wherein the product comprises a liquid-permeable surface material and a backside material that is liquid-tight, wherein the absorption body is arranged between the liquid-permeable surface material and the liquid-tight backsheet material, and wherein the liquid-receiving, open-cell foam layer is placed against the liquid-permeable surface material and the liquid-absorbent, fibrous layer is placed against the liquid-tight backsheet material.

11. The absorbent product according to claim 1, wherein the product further comprises a front part, a back part and a crotch part located between the back part and the front part, wherein the liquid-absorbent, fibrous layer extends in the longitudinal direction of the product over the crotch part and at least a portion over the front part, wherein the product has a width in the transition between the crotch part and the front part which is less than 40 mm, and wherein the side edges of the liquid-absorbent, fibrous layer diverge in the direction from the transition between the crotch part and the front part to at least a portion over the front part.

12. The absorbent product according to claim 1, wherein the absorbent product is a sanitary napkin, a panty liner or an incontinence protector.
